(19) United States
(12) Patent Application Publication
(10) Pub. No.: US 2024/0054700 A1
(43) Pub. Date: Feb. 15, 2024

(54) POST-PROCESSING FOR RADIOLOGICAL IMAGES

(71) Applicant: KONINKLIJKE PHILIPS N.V., EINDHOVEN (NL)

(72) Inventors: CHARLES LOEB TRUWIT, WAYZATA, MN (US); HANS-ALOYS WISCHMANN, HENSTEDT-ULZBURG (DE); MERLIJN SEVENSTER, HAARLEM (NL); HILDO LAMB, EINDHOVEN (NL)

(21) Appl. No.: 18/270,892

(22) PCT Filed: Jan. 12, 2022
(86) PCT No.: PCT/EP2022/050509

§ 371 Date: Jul. 5, 2023

Related U.S. Application Data
(60) Provisional application No. 63/138,087, filed on Jan. 15, 2021.
(60) Provisional application No. 63/296,221, filed on Jan. 4, 2022.

(51) Int. Cl. G06T 11/00; G06T 7/00; G06T 11/60; G06F 3/04845; G06F 3/0481; G16H 30/20; G16H 10/60; G16H 30/40; A61B 6/00

(52) U.S. Cl. CPC G06T 11/008; G06T 11/006; G06T 7/0012; G06T 11/60; G06F 3/04845; G06F 3/0481; G16H 30/20; G16H 10/60; G16H 30/40; A61B 6/469; A61B 6/463; G06T 2211/441; G06T 2200/24; G06T 2207/10081; G06T 2207/10088; G06T 2207/10104; G06T 2207/30101; G06T 2207/30064; G06T 2210/41; G06T 2210/62; G06F 2203/04804

(57) ABSTRACT

A computer (110)-implemented method for reading an imaging scan (410) includes accessing the imaging scan (410). The imaging scan (410) includes a stack of radiological images. The method also includes generating a plurality of two-dimensional images from cross-sectional data of the imaging scan (410). The plurality of two-dimensional images include projected information from the stack of radiological images. The projected information includes either a full imaged volume or an automatically-selected sub-volume and either a full range of image intensities or an automatically-selected sub-range of image intensities. The method further includes displaying the generated plurality of two-dimensional images or a subset thereof in a user interface (UI) of an advanced interpretation environment (380). The user interface provides access to the stack of radiological images or additional information derived from the stack of radiological images, by enabling interaction with the generated plurality of two-dimensional images.

### CROSS-REFERENCE TO THE RELATED APPLICATION

This international patent application claims the benefit of priority under 35 U.S.C. § 119(e) to U.S. Provisional Application No. 63/138,087, filed on Jan. 15, 2021 in the United States Patent and Trademark Office, the contents of which are herein incorporated by reference in their entirety.

### BACKGROUND

### 1. Field

The present disclosure relates to workflow management for reading three-dimensional radiological imaging exams including screening workflows and diagnostic clinical workflows.

### 2. Description of the Related Art

Chest x-ray (CXR) is the most common radiological exam type, accounting for roughly half of all radiological examinations performed globally. CXR acquisition is fast and exposes the patient to a small X-Ray dose (˜0.1 mSv). CXRs are acquired by means of diagnostic X-Ray “Bucky” systems that are cost efficient compared to other imaging modalities such as magnetic resonance imaging (MRI) and computed tomography (CT). CXRs provide significant, but limited, diagnostic value. When a radiologist identifies a suspect finding on CXR, oftentimes an appropriate follow-up examination, such as a computed tomography (CT) or positron emission tomography (PET or PET-CT) study. is ordered to assess the finding more definitively.

Acquisition of regular CT images typically exposes the patient to considerably higher radiation doses than CXRs (˜7 mSv), while yielding cross-sectional images of superior diagnostic value as compared to CXRs. To avoid the increased dose exposure, CT protocols are being designed to use low and ultra-low radiation doses. For lung cancer screening, so-called low-dose (LDCT) images, and more recently ultra-low-dose CT (ULDCT) images, may be acquired (˜1 mSv or less), resulting in images that retain sufficient image quality and diagnostic value for the screening purpose at hand.

In a recent study (Kroft, et al., Journal of Thoracic Imaging, 2019, 179-186), the diagnostic value of C×R images and ULDCT images were compared in 200 patients. Participating patients received a CXR study and an ULDCT study on the same day. The same radiologist first read the CXR study, then the ULDCT study. In 40 of the 200 patients, the ULDCT findings impacted management of care as compared to the care path that would have been initiated based on the CXR findings alone. The management of care is improved, for example, based on either or both of confirmation of newly detected findings and/or findings that were determined not to be present or findings that were determined to be present but insignificant, due to the ULDCT findings. The diagnostic confidence was significantly higher for radiologists interpreting ULDCTs than for CXRs, while radiation doses (0.07 mSv versus 0.04 mSv, respectively) and in-room times were comparable.

ULDCT may be superior to CXR in certain aspects, and comparable in many others. However, radiologist interpretation time for CTs is considerably higher than radiologist interpretation time is for CXRs. CT interpretation may take ten (10) to fifteen (15) minutes, in contrast to the one (1) to three (3) minutes that is typical for CXR interpretation, depending on the case complexity and patient imaging history. Even though widespread adoption of ULDCT promises to improve the standard of care, ULDCT may at the same time increase radiologist reading times by as much as a factor of three to five. Such a large increase in reading times cannot be practically absorbed by the current (and future) radiologist workforce, which is already subject to severe overload and high burnout rates.

### SUMMARY

According to an aspect of the present disclosure, a computer-implemented method for reading an imaging scan includes accessing the imaging scan. The imaging scan includes a stack of radiological images. The method also includes generating a plurality of two-dimensional images from cross-sectional data of the imaging scan. The plurality of two-dimensional images include projected information from the stack of radiological images. The projected information includes either a full imaged volume or an automatically-selected sub-volume and either a full range of image intensities or an automatically-selected sub-range of image intensities. The method further includes displaying the generated plurality of two-dimensional images or a subset thereof in a user interface (UI) of an advanced interpretation environment. The user interface provides access to the stack of radiological images or additional information derived from the stack of radiological images, by enabling interaction with the generated plurality of two-dimensional images.

According to another aspect of the present disclosure, a system for reading an imaging scan includes a memory that stores instructions, and a processor that executes the instructions. When executed by the processor, the instructions cause the system to access the imaging scan. The imaging scan includes a stack of radiological images. The instructions also cause the system to generate a plurality of two-dimensional images from cross-sectional data of the imaging scan. The plurality of two-dimensional images include projected information from the stack of radiological images. The projected information includes either a full imaged volume or an automatically-selected sub-volume and either a full range of image intensities or an automatically-selected sub-range of image intensities. The instructions further cause the system to display the generated plurality of two-dimensional images or a subset thereof in a user interface (UI) of an advanced interpretation environment. The user interface provides access to the stack of radiological images or additional information derived from the stack of radiological images, by enabling interaction with the generated plurality of two-dimensional images.

According to another aspect of the present disclosure, a controller for reading an imaging scan includes a memory that stores instructions, and a processor that executes the instructions. When executed by the processor, the instructions cause a system which includes the controller to access the imaging scan. The imaging scan includes a stack of radiological images. The instructions also cause the system to generate a plurality of two-dimensional images from cross-sectional data of the imaging scan. The plurality of two-dimensional images include projected information from the stack of radiological images. The projected information includes either a full imaged volume or an automatically-selected sub-volume and either a full range of image intensities or an automatically-selected sub-range of image intensities. The instructions further cause the system to display on the display the generated plurality of two-dimensional images or a subset thereof in a user interface (UI) of an advanced interpretation environment. The user interface provides access to the stack of radiological images or additional information derived from the stack of radiological images, by enabling interaction with the generated plurality of two-dimensional images.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for post-processing for radiological images, in accordance with a representative embodiment.

FIG. 1B illustrates a controller for post-processing for radiological images, in accordance with a representative embodiment.

FIG. 2 illustrates a method for post-processing for radiological images, in accordance with a representative embodiment.

FIG. 3 illustrates an environment architecture for post-processing for radiological images, in accordance with a representative embodiment.

FIG. 4 illustrates a view flow for post-processing for radiological images, in accordance with a representative embodiment.

FIG. 5 illustrates ROI-based artifact detection for post-processing for radiological images, in accordance with a representative embodiment.

FIG. 6 illustrates a subspace viewer for post-processing for radiological images, in accordance with a representative embodiment.

FIG. 7 illustrates a user interface for post-processing for radiological images, in accordance with a representative embodiment.

FIG. 8 illustrates a computer system, on which a method for post-processing for radiological images is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

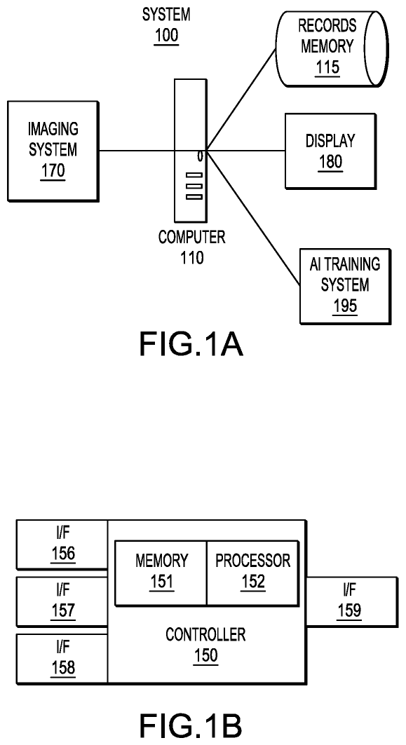

FIG. 1A illustrates a system 100 for post-processing for radiological images, in accordance with a representative embodiment.

The system 100 in FIG. 1A is a system for post-processing for radiological images and includes components that may be provided together or that may be distributed. The system 100 includes a computer 110, a records memory 115, an imaging system 170, a display 180 and an AI training system 195 (artificial intelligence training system).

The computer 110 may have a controller 150 as shown in FIG. 1B and as explained below. The computer 110 is provided access to an imaging scan from the imaging system 170 either directly or indirectly. The computer 110 is configured to access the imaging scan, either from the imaging system 170 directly or from an intermediate storage and/or processing system that preprocesses the imaging scan. The imaging scan includes a stack of radiological images. The computer 110 is also configured to generate multiple two-dimensional images from cross-sectional data of the imaging scan. The plurality of two-dimensional images include projected information from the stack of radiological images. The projected information comprises either a full imaged volume or an automatically-selected sub-volume and either a full range of image intensities or an automatically-selected sub-range of image intensities. The computer 110 may also be configured to generate or to control generation of a display which is displayed on the display 180.

The computer 110 may store and execute sub-modules to process the cross-sectional data. Different sub-modules may be used to process different instances of cross-sectional data, such as based on patient data, disease data, imaging modality data, and so on. The processing by different sub-modules may result in presentation of variable types of two-dimensional images. Variations in two-dimensional images may include editable features such as those that highlight, accentuate, or suppress fat, bone and/or (soft) tissue that can be edited out of two-dimensional images depending on the data provided to the sub-module which is processing the cross-sectional data.

The records memory 115 is representative of a memory system that stores records such as profile data objects corresponding to patients of imaging scans. Profile data objects may comprise patient data. Patient data may be provided from one or more of a radiology information system (RIS), an HL7 broker (health level seven broker), or an electronic medical record (EMR) system. HL7 refers to a set of international standards for transfer of clinical and administrative data between healthcare software applications. The patient data which may be used as the basis for selecting a sub-module may be patient data from the records memory 115, or meta information extracted from the (DICOM) image data provided by the imaging system 170.

The imaging system 170 may be a computed tomography imaging system, such as an ULDCT. However, the applications of the post-processing for radiological images described herein are not limited to ULDCT or even to CT generally. Nor are applications of the post-processing for radiological images limited to imaging of the chest (thorax), as the teachings described herein may be applied to imaging of a variety of types of anatomy. Examples of the imaging system 170 include, but are not limited to, a so-called conventional CT imaging system which produces a conventional CT scan, LDCT, or ULDCT scan. Additionally, other than conventional CT imaging systems, such as a dual energy CT imaging system, a spectral CT imaging system, a dark field CT imaging system and a photon counting CT imaging system, or some combination of these imaging systems, can all be used to generate a CT, a LDCT and/or an ULDCT, as described above and therefore may serve as the imaging system 170 in FIG. 1A, to generate images consistent with the teachings described herein. Additionally, a magnetic resonance (MR) imaging system which produces a MR scan, or a PET or combined PET-CT imaging system which produces a positron emission tomography (PET) scan may serve as the imaging system 170 in FIG. 1A.

The display 180 may be local to the computer 110 or may be remotely connected to the computer 110 via a standard web interface. The display 180 may be connected to the computer 110 via a local wired interface such as an Ethernet cable or via a local wireless interface such as a Wi-Fi connection. The display 180 may be interfaced with other user input devices by which users can input instructions, including mouses, keyboards, thumbwheels and so on.

The display 180 may be a monitor such as a computer monitor, a display on a mobile device, an augmented reality display, a television or projection device, an electronic whiteboard, or another screen configured to display electronic imagery. The display 180 may also include one or more input interface(s) that may connect other elements or components to the computer 110, as well as an interactive touch screen configured to display prompts to users and collect touch input from users.

The AI training system 195 is representative of a system that trains artificial intelligence applied by the computer 110. Trained AI may be applied to generate the plurality of two-dimensional images, to edit the plurality of two-dimensional images, and/or to vary the plurality of two-dimensional images. Trained AI may also be applied to analyze the stack of radiological images so as to generate, edit and/or vary the plurality of two-dimensional images. The trained AI may be used to execute one or more artificial intelligence processes on the stack of radiological images. Examples of functions performed using such artificial intelligence processes include, but are not limited to, detecting anatomical characteristics in the stack of radiological images. Projected information from the stack of radiological images may be derived based on an anatomical characteristic detected from the one or more artificial intelligence processes. AI may include Machine Learning (ML) and specifically Deep Learning (DL) methods, but also or instead traditional statistical methods and/or rule-based engines derived from clinical and/or workflow knowledge.

FIG. 1B illustrates controller 150 for post-processing for radiological images, in accordance with a representative embodiment.

The controller 150 includes a memory 151, a processor 152, a first interface 156, a second interface 157, a third interface 158, and a fourth interface 159. The memory 151 stores instructions which are executed by the processor 152. The processor 152 executes the instructions. The controller 150 may be provided in the computer 110, though the controller 150 may alternatively be provided as a stand-alone controller.

The first interface 156, the second interface 157, the third interface 158 and the fourth interface 159 may include ports, disk drives, wireless antennas, or other types of receiver circuitry. The first interface 156, the second interface 157, the third interface 158 and/or the fourth interface 159 may connect the computer 110 to the records memory 115, the imaging system 170, the display 180 and the AI training system 195.

The controller 150 may perform some of the operations described herein directly and may implement other operations described herein indirectly. For example, the controller 150 may directly or indirectly access the imaging scan from the imaging system 170 by executing an instruction to retrieve the imaging scan. The controller 150 may also directly generate the plurality of two-dimensional images from cross-sectional data of the imaging scan. The controller 150 may indirectly control operations such as by generating and transmitting content to be displayed on the display 180. Accordingly, the processes implemented by the controller 150 when the processor 152 executes instructions from the memory 151 may include steps not directly performed by the controller 150.

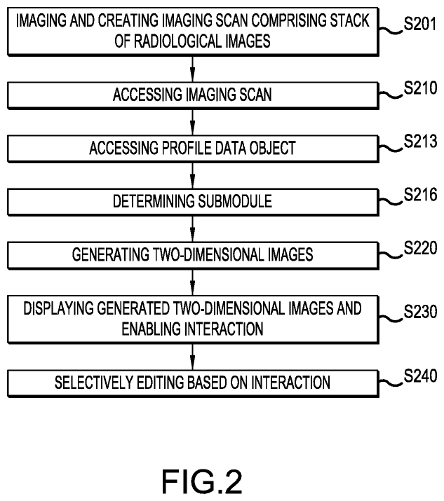

FIG. 2 illustrates a method for post-processing for radiological images, in accordance with a representative embodiment.

The method of FIG. 2 may be performed by the system 100 including the computer 110 and the display 180.

At S201, the method of FIG. 2 includes performing imaging and creating the imaging scan comprising the stack of radiological images. The imaging and creation of the imaging scan may be performed by the imaging system 170 as a precursor for the post-processing for radiological images described herein. For example, the imaging system 170 may perform imaging and create the imaging scan, and then store the images in an intermediate medical imaging system or a Picture Archive and Communication System (PACS), either of which is accessible to the computer 110.

At S210, the method of FIG. 2 includes accessing the imaging scan. The imaging scan includes a stack of radiological images. The accessing of the imaging scan may be performed by the computer 110 in FIG. 1A. The imaging scan accessed at S210 may be, for example, any of a CT scan, a LDCT, an ULDCT scan, a spectral or dual energy CT scan, a MR scan, or a PET or PET-CT scan.

At S213, the method of FIG. 2 includes accessing a profile data object. The profile data object may be accessed by the computer 110 from the records memory 115 in FIG. 1A and/or the meta information in the (DICOM) image data. The profile data object may correspond to a patient of the imaging scan. The profile data object may include patient data from one or more of a Radiology Information System (RIS), an HL7 broker, or an Electronic Medical Record (EMR) system.

At S216, the method of FIG. 2 includes determining a submodule. The determined submodule may be stored in and retrieved from the memory 151 among a plurality of submodules. The submodule may be selected to process the cross-sectional data of the imaging scan based on the accessed profile data object. Different submodules may generate different types and sets of two-dimensional images based on the profile data object.

At S220, the method of FIG. 2 includes generating two-dimensional images. The plurality of two-dimensional images are generated from cross-sectional data of the imaging scan. The plurality of two-dimensional images comprise projected information from the stack of radiological images. The projected information includes either a full volume covered by the stack of radiological images or an automatically-selected sub-volume and either a full range of image intensities contained in the stack of radiological images or an automatically-selected sub-range of image intensities, e.g. for a stack of CT images only Hounsfield values that correspond to soft tissue or only Hounsfield values that correspond to bones. In addition to varying the processing based on the submodule selected based on the accessed profile data object, the plurality of two-dimensional images may be selectively editable. The plurality of two-dimensional images may be used to partly reconstruct or otherwise recreate the imaging scan with and without various features captured in the imaging scan. Reconstructed images may be generated synthetically from the stack of radiological images and based on one or more detected an anatomical characteristic(s). The two-dimensional images may be reconstruction images generated synthetically from the stack of radiological images and based on the detected anatomical characteristic(s).

In some embodiments, generating the two-dimensional images at S220 may include executing one or more AI processes on the stack of radiological images. One or more AI processes may be configured to detect anatomical characteristics in the stack of radiological images. As an example, the projected information from the stack of radiological images may be derived based on an anatomical characteristic detected from the one or more AI processes. Examples of such detected anatomical characteristics include lesions, or types of anatomy such as bone or tissue, or types of anatomy such as organs, vessels/vasculature tree, or bronchi or airways/respiratory tree.

At S230, the method of FIG. 2 includes displaying the generated plurality of two-dimensional images or a subset thereof and enabling interaction. The displayed plurality of two-dimensional images may be displayed on the display 180. The plurality of two-dimensional images may be interactively selected and edited, such as to show particular features present in certain pairs or triplets of two-dimensional images but not in other pairs or triplets of two-dimensional images. As one example, one pair or triplet of two-dimensional images may suppress visualization of bones captured in the imaging scan, such as when display of the bones is a form of irrelevant noise on the plurality of two-dimensional images relative to the task at hand. Similarly, pairs or triplets of two-dimensional images may suppress visualization of tissue when display of the (soft) tissue is a form of irrelevant noise on the plurality of two-dimensional images relative to the task at hand.

Examples of information displayed on the display 180 with the two-dimensional images include a list of one or more measurements, such as measurements of a detected lesion. Such measurements may flag an incidental finding, and may link to an image location corresponding to a region of interest indicator. Incidental findings may be findings that are seen because they are located within the field of view, but not related to the indication for scan—e.g., a potentially malignant lung nodule seen on a coronary CT angiogram. Other examples of information displayed on the display 180 may include a semi-transparent rendering of an object of interest overlaid on one or more of the plurality of two-dimensional images. An object of interest may be one of lungs, a vasculature tree, or a respiratory tree.

At S240, the method of FIG. 2 includes selectively editing the generated plurality of two-dimensional images or a subset thereof based on interaction. For example, the selective editing at S240 may include selectively editing an object captured in the stack of radiological images. The plurality of two-dimensional images may be editable to selectively display fewer than all types of anatomy captured in the stack of radiological images. At least one of the plurality of two-dimensional images generated at S220 may include the selectively-edited object. A displayed pair of two-dimensional images or triplet of two-dimensional images may be selectively edited to remove bone or tissue based on an instruction accepted from a user via a user interface. For example, a pair of two-dimensional images may display the posterior-anterior and lateral (PA+LAT) views, whereas a triplet of two-dimensional images may remove the overlay of the left and right hemithoraces typical of a single LAT. Remembering that left and right are swapped in the typical PA view (as though looking at a photograph taken from in front of the patient, thereby “seeing” the left arm at the far right of the photograph), in the triplet display, the “left half” lateral projection from the centerline might be displayed further to the right of the PA view, thus aligning the left hemithorax on the PA with the left hemithorax LAT, plus the “right half” LAT adjacent and to the left of the right hemithorax on the PA. In other examples, pairs or triplets of two-dimensional images may be removed from a display and replaced by other pairs or triplets of two-dimensional images as a form of editing based on an instruction accepted from a user via a user interface. The selective editing may be performed by the computer 110 and reflected in the plurality of two-dimensional images displayed on the display 180.

An advanced reading environment in which post-processing of radiological images may be used provides, for example, augmentations, annotations, and visualizations which may enable radiologists to efficiently read three-dimensional CT, LDCT and ULDCT studies. Efficiencies may be gained by focusing inspection on a limited number of two-dimensional CXR-like views automatically created from the cross-sectional image data. Model-based and AI-enabled tools may extract and present relevant information from the underlying full three-dimensional ULDCT study as separate views and/or overlays. As a result, the high diagnostic value of ULDCT may be obtained at radiologist reading times comparable to CXRs.

Additionally, the augmented reading environment described herein benefits radiologist interpretation of normal and low-dose CTs, without and with contrast, and without or with spectral or dual energy information.

As described herein, conversion from 3D ULDCT image data to a 2D projection in posterior-anterior (PA) direction simulates a conventional PA chest X-Ray. Furthermore, 3D information from ULDCT allows reconstruction of 2D lateral views of right and left lung separately. This approach adds extra views with clinical value as compared to a single lateral view which is commonly acquired with conventional chest X-Ray, where right and left lung are projected on top of each other and cannot be evaluated separately. Combined with features such as AI-based detection of anatomical characteristics and selective editing of the generated plurality of two-dimensional images, a radiologist is enabled to quickly process results of an imaging scan comprising a stack of radiological images.

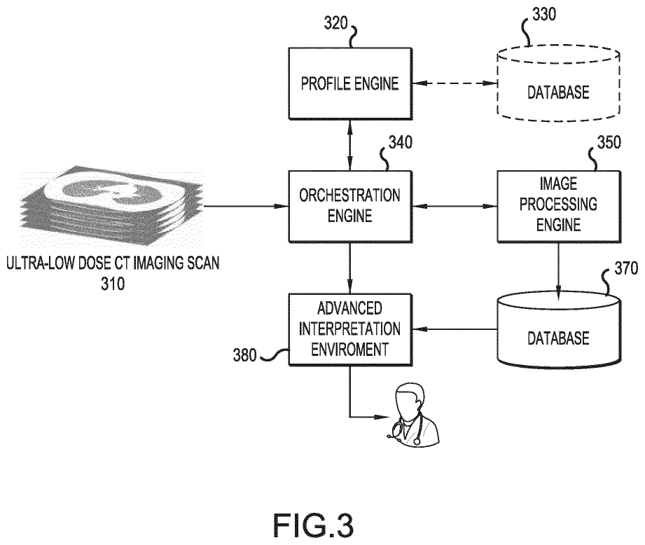

FIG. 3 illustrates an environment architecture for post-processing for radiological images, in accordance with a representative embodiment.

In the example shown in FIG. 3, the augmented reading environment includes an environment architecture which includes a profile engine 320, a database 330, an orchestration engine 340, an image processing engine 350, and an advanced interpretation environment 380.

The profile engine 320 obtains relevant meta-data about a multi-dimensional radiological image stack and the patient. As an example, the image stack may be a DICOM (digital imaging and communications in medicine) image set, such as an ULDCT DICOM image set. The profile engine 320 stores and retrieves the relevant meta-data to and from the database 330. The orchestration engine 340 receives the multi-dimensional radiological image stack and routes the DICOM image set to subsequent image processing modules of the image processing engine 350 based on the input received from the profile engine 320. The image processing modules of the image processing engine 350 process the multi-dimensional radiological image stack and derive additional images, views, and analyses from the input DICOM image set.

The database 370 stores the output of the image processing engine 350 and the original images. The advanced interpretation environment 380 is a DICOM viewing environment in which radiologists may view the ULDCT images, views, and analyses created by the image processing engine 350. The advanced interpretation environment 380 implements user interface (UI) features for efficient interaction with the images, views, and analyses.

The profile engine 320 receives the ULDCT DICOM image and extracts metadata about the examination and about the patient. The extracted metadata may then be enriched with available information from the RIS/PACS (radiology information system/picture archiving and communication system) and/or EMR system. The profile engine 320 may be configured to extract specified metadata according to a data object (e.g., XML, JSON, etc.) in which each field is tagged with the information type contained in the field. Examples of metadata which may be extracted by the profile engine 320 include, for example, contrast use indications, clinical questions, cancer indications, diabetes indications, and symptom indications such as fever, etc.

The profile engine 320 may pull examination metadata from the DICOM image, such as, for example, information about the use of contrast medium and respective amounts. As per the DICOM standard, examination metadata is stored in controlled fields with unambiguously pre-defined semantics.

Relevant data about the patient may be pulled from select fields from the DICOM image itself and, optionally, from relevant hospital information technology (IT) systems, such as the radiology information system, for symptoms and reason for exam; an HL7 broker, for recent lab values, clinical notes, recent radiology, cardiology, and pathology reports; and/or Electronic Medical Records (EMRs) system, for recent lab values, clinical notes, recent radiology, cardiology, and pathology reports, medications, and co-morbidities).

Depending on the nature of the information that is received from these systems, dedicated software may be used to normalize its contents. For instance, for free-text documents (e.g., notes, reports, etc.), natural language processing (NLP) technology may be used to impose a structure on these documents (e.g., section-subsection-paragraph-sentence), extract concepts from a controlled vocabulary or ontology (e.g., SNOMED, RadLex, etc.), extract negations, and feed modules that search for positive occurrences of diagnoses. Other information sources (e.g., lab values, problem lists, etc.) may include lists of elements from controlled vocabularies (e.g., LOINC, ICD-10, etc.) and numeric values.

The orchestration engine 340 receives an ULDCT DICOM image, sends the ULDCT DICOM image to the profile engine 320, and receives a profile data object. For example, the orchestration engine 340 may receive the ULDCT DICOM image from the modality/scanner or from an image router or from a picture archive and communication system. The orchestration engine 340 may then send the ULDCT DICOM image to modules in the image processing engine 350, depending on the information in the profile data object. The orchestration engine 340 has access to a rule base, either manually coded or created using AI tools, with each rule mapping information in the profile data object onto one or more modules in the image processing engine 350. For instance, Rule 1 below is an example of a rule for applying a pulmonary embolism detection module where a corresponding patient of a received ULDCT DICOM image is older than eighteen (18) and contrast was used in the examination.

(patient_has_age>18)&&(contrast_use==yes)=>apply module “detect_pulmonary_embolism” Rule 1

The rule base may also contain rules that have an empty antecedent. Such rules apply to each and every profile data object and thus always trigger the module.

The image processing engine 350 includes one or more submodules which each create a new respective image, view, or analysis. The output of these submodules may be added to the ULDCT DICOM image as a secondary capture (e.g., a new series in the DICOM image) or stored in the database 370 in the appropriate format (e.g., jpg or DICOM overlay, DICOM-SR, free-text, etc.).

The submodules of the image processing engine 350 are implemented using techniques, such as projections, advanced visualization, and (AI-enabled) computer-aided detection for augmentation and annotation. The submodules may be applied serially, or in other meaningful combinations.

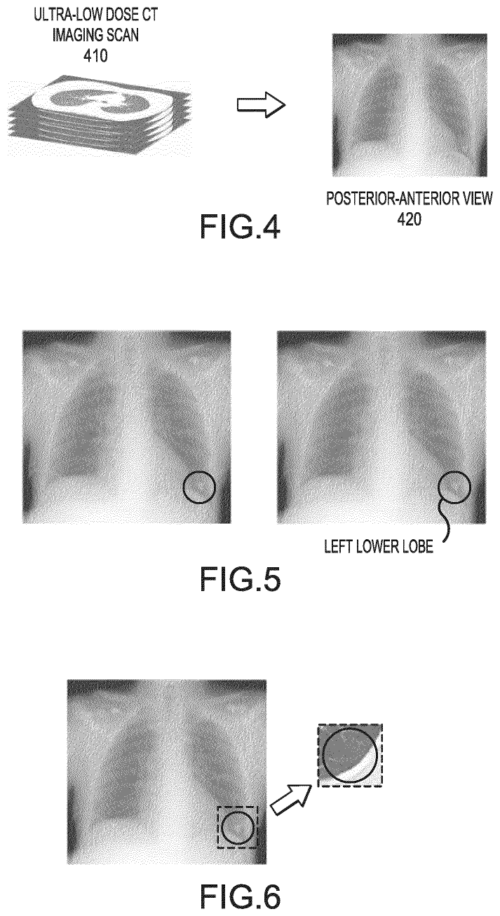

Radiologists are highly skilled at instant review of two-dimensional CXR views, predominantly posterior-anterior and lateral views. These views may be reconstructed synthetically from the ULDCT image using well known in the art techniques. In addition, using the same techniques, other non-standard views may be created from the ULDCT image. FIG. 4. illustrates a standard PA view (DRR) computed from a stack of radiological images. Non-standard views may also be recreated from the stack of radiological images. Examples of non-standard views that may be computed from stacks of radiological images consistent with the teachings herein include, but are not limited to, two-dimensional projection images of the left and/or right hemithorax, two-dimensional images in which bones are not present after being segmented and removed in three dimensions, and other non-standard two-dimensional views.

FIG. 4 illustrates a view flow for post-processing for radiological images, in accordance with a representative embodiment.

In FIG. 4, an ultra-low dose CT imaging scan 410 is an imaging scan comprising a stack of radiological images. The ultra-low dose CT imaging scan 410 is used to generate a plurality of two-dimensional images from cross-sectional data of the ultra-low dose CT imaging scan 410. The two-dimensional images include the posterior-anterior view 420.

Advanced Visualization (AV) techniques may be applied to the ultra-low dose CT imaging scan 410 which is three-dimensional to create views on the image data. Instances of relevant AV include segmentation and visualization of the vascular tree, of the respiratory or bronchial tree, of select organs, and of ribs, vertebrae, and other bones, etc. Segmentation and visualization of the vascular tree may be applied if contrast was used in the imaging. Examples of select organs to which segmentation and visualization or suppression may be applied include, but are not limited to, heart, ribs, lungs, lung lobes etc.

The three-dimensional AV techniques may be applied to include or exclude—and hence focus on or suppress—certain anatomies (e.g., lungs, heart, ribs, vertebrae, etc.) and then to create standard posterior-anterior (PA) and lateral CXR-like views to obtain a clear two-dimensional view of the data. As a result, rib suppression techniques may be implemented, for example, by first removing the segmented ribs in three dimensions from the image and then creating the standard two-dimensional PA and lateral views.

The segmentations or overlays created by the AV techniques may also be used to create measurements, such as aortic measurements. These measurements may themselves be projected as a view in the DICOM images (e.g., as annotations, a jpeg, etc.) or as a separate file (e.g., as .txt, .csv, etc.) in the database 370, and may be used to identify incidental findings to highlight to the radiologist, such as when they are out of bounds with respect to normative values.

Multiple of the advanced two-dimensional visualizations and/or projections may be picked up by the advanced interpretation environment 380, as described below, and may represent a key aspect for driving efficiency in the radiologist reading and interpretation workflow.

AI techniques currently in the art may be used to automatically detect lesions (e.g., fractures, malignancies, pulmonary embolism, etc.) on CT images. Such AI techniques may be applied in three-dimensions to the ULDCT image set for automated detection of relevant findings. Moreover, AI techniques may be applied to the reconstructed two-dimensional views reconstructed from the ULDCT, in some cases after a transfer learning step, as the computed “CXR”-like projections show different noise statistics (and thus different signal-to-noise ratio) than standard C×R images.

In one example, a model-based or AI-enabled segmentation may be applied to the ULDCT to label all voxels belonging to one of the organs and/or tissue types detected by the model, such as heart, lungs, ribs, etc. The segmentation model may have arbitrary granularity, so that, by means of example, the AI is able to detect lung voxels as well as voxels belonging to the inferior or lower aspect of the lung or the left lower lobe.

In one example, the AI may draw a region of interest (ROI) around detected lesions and annotate them with the anatomical location derived from the three-dimensional image set. The regions of interest may be positioned within the three-dimensional segmentation model adapted to the respective scan. Further, the anatomical location may be extracted automatically for each AI detected lesion. For example, AI may derive that a lung nodule is located within the left lower lobe (inferior or lower aspect of the lung).

Similarly, the AI may draw a region of interest around detected artifacts and annotate them with the anatomical location derived from the three-dimensional image set. For example, imaging artifacts such as electrocardiogram (ECG) leads being included in an image, may be detected.

FIG. 5 illustrates ROI-based artifact detection for post-processing for radiological images, in accordance with a representative embodiment.

Here, ECG lead artifacts have been detected and highlighted in one image and, in the other image, the ECG lead in the left lower lobe (inferior or lower aspect of the lung) has been labelled according to the region of interest in which the ECG lead is detected.

Two-dimensional views may be created from the ULDCT image and the AI-detected regions of interest and locations of the AI-detected regions of interest may be associated with the ULDCT as metadata. The associated metadata may include pixel regions on the two-dimensional views. Results of quantification may also be displayed, for example, as standard volumes or diameters that may only be calculated in the three-dimensional image data.

The advanced interpretation environment 380 includes a standard DICOM viewer able to show the ULDCT DICOM image, secondary captures, and views reconstructed from the ULDCT. In addition to standard image viewing capabilities, the advanced interpretation environment 380 implements advanced user interaction principles that allow a radiologist to interrogate the three-dimensional ULDCT image through a limited number of two-dimensional views and interactions. Interactions may include rotations, localizations, and focus on a subspace.

The radiologist may create new views as needed or preferred by rotating pre-computed views (e.g., rotating away from standard PA or lateral views). For a joint image, this may provide a post-acquisition angulation, such as in the case where the joint was not, or could not be, imaged in a standard pose. For an image of the spine, issues with a rotated pose of the patient standing in front of the X-Ray detector may be alleviated by reorienting the projection direction for the creation of the two-dimensional image(s) instead of requiring a retake.

The radiologist may request the exact location of an AI-detected region of interest through user interface interactions, such as by particular mouse and/or keyboard shortcuts. In effect, the radiologist may quickly and efficiently explore detected regions of interest with minimal navigation through interfaces, options, menus, selecting/clicking on an object or region of interest, and the like.

The radiologist may view a two-dimensional region of interest in three-dimensions. For example, the radiologist may draw a circle around a specific region and then select (e.g. from a drop-down menu, context menu, etc.) an option labelled “Inspect two-dimensional plane in three dimensions” or the like. In response, a DICOM view may be generated and provided to the radiologist (e.g., via pop up, etc.) that shows the region on the ULDCT in a traditional coronal or sagittal view. When the radiologist draws a region of interest circle around an AI-detected finding, the “Inspect in three-dimensions” option may limit the displayed three-dimensional subspace to an area around the lesion, including three (3) orthogonal (e.g., CC, coronal, and sagittal) slices intersecting at the object of interest.

FIG. 6 illustrates a subspace viewer for post-processing for radiological images, in accordance with a representative embodiment.

FIG. 6 shows an example of the subspace view displaying a traditional coronal view in response to a subspace selection. In FIG. 6, the inset includes a nodule that may or may not be visible on the main 2D projection, where the AI described herein may detect the nodule and make the region shown in the inset selectable as a region of interest based on detecting the nodule.

The subspace viewer provides multiple benefits. Spatial localization by the reader is improved as the reader may quickly verify where within the three-dimensional space of the body a region of interest is located. For example, the reader may check whether a detected lesion is well within the lung or attached to the chest wall. Moreover, inspection through the subspace viewer may reveal additional characteristics to improve overall analysis, such as determining whether a lesion is likely malignant or benign or enabling or prompting radiomics to suggest a tumor subtype (which may then be used to suggest appropriate therapy, etc.).

In order to enable radiological interpretation of three-dimensional ULDCT data stacks within a time frame that is similar to that for reading pairs of two-dimensional Chest X-Ray (CXR) images, the above features may be combined in an automated or guided workflow.

A three-dimensional ULDCT is acquired. The orchestration engine 340 in FIG. 3 may then leverage meta information from the profile engine 320 FIG. 3 to apply the appropriate set or sequence of submodules from the image processing engine 350 in FIG. 3, including any relevant AI submodules. A resultant set of augmented, annotated, two-dimensional pseudo-C×R images, which carry the full three-dimensional information but show only two-dimensional projections may then be viewed by a radiologist.

The advanced interpretation environment 380 in FIG. 3 shows the radiologist standard and processed PA and lateral projections. The standard projections may include two (2) images or three (3) images calculated from the three-dimensional ULDCT (“raw”) image data. Two (2) images may correspond to a traditional pair of PA and lateral projections, and three (3) images may include a partial Left lateral and a partial Right lateral projection, from a central plane.

The processed projections may include two (2) images or three (3) images that include highlights of any AI findings from the background processing. The radiologist may accept, reject, or more deeply inspect the two-dimensional views of the processed projections by rotating the two-dimensional view or by stepping into the full three-dimensional cross-sectional views. The AI findings may be generated by background processing on the three-dimensional data as well as on the standard two-dimensional projections.

The advanced interpretation environment 380 may also show the radiologist projections, or semi-transparent renderings of objects of interest, that may include relevant measurements as annotations from the three-dimensional segmentations. The object of interest may include a semi-transparent view of one of lungs, a vasculature tree and/or a respiratory tree, plus any suspicious findings.

Moreover, the advanced interpretation environment 380 may display lists of measurements. Incidental findings may be flagged and be associated with links to image locations for review via the processed projections features described above.

The two-dimensional images may be inspected as static projections, without requiring any further interaction, and with improved signal-to-noise ratio because they are calculated from scatter-free CT images and structures, such as bones and other noisy anatomical elements, have been removed or isolated in accordance with the anatomy being examined. A level and window setting may be automatically set for each two-dimensional pseudo-C×R image pair or triplet, and zoom and crop may be automatically set for anatomies of interest in accordance with the imaging request or clinical question, or to highlight suspicious regions of interest. Setting the level and window may include selecting a subset of gray values by specifying the level (e.g., a midpoint value) and the window (e.g., a width value) of the gray values that may then be stretched to the full black-white range to maximize relevant contrast.

As a result, unnecessary scrolling through large portions of ULDCT slices, often in multiple view modes, may be avoided. Instead, a low number of two-dimensional images may be inspected. The two-dimensional images may be further explored in a focused manner by interacting with the user interface and rotating as desired. Simultaneously, model-based and AI-enabled background processes operating on the full stack of ULDCT slices increase quality, accuracy, and value of the reading. Pseudo-X-Ray visualization adheres much more closely to existing image reading paradigms, and thus leverages already established experiences of radiologists, and existing textbook knowledge, more readily.

The described methods and systems may also be leveraged to support contrast and non-contrast dual-energy or spectral CT, as well as next-generation spectral CT, sometimes referred to as photon-counting CT. In such cases, a three-dimensional segmentation of structures based on the material properties of the structures may serve to visualize and read only rotatable two-dimensional projections of physical properties, rather than of three-dimensional absorption values or of three-dimensional material/property maps. In some examples, the ULDCT may be combined with a low, or very low, volume of contrast agent. Additionally, “always on” current and future spectral CT may enable segmentation of the vascular tree even without contrast agent, in which case the systems and methods described herein may further save time as every scan must be read for findings in the vasculature tree, and all relevant measurements must be taken.

A number of other applications are possible, including longitudinal follow-up comparison of “two-dimensional X-Rays” (e.g., for joints, fractures, etc.) and/or longitudinal tracking of disease progression in organs such as, for example, the lungs and the like (e.g., COVID, pneumonia, etc.) where inhalation status and pose must also be normalized and/or registered in the three-dimensional space. These other applications may otherwise be very difficult due to changes in pose and the like in two-dimensional, but which may be more feasible via elastic registration in three-dimensions, whereas pseudo-CXR-images, generated by the above-described systems and methods, match the prior image data exactly in position and grayscale values.

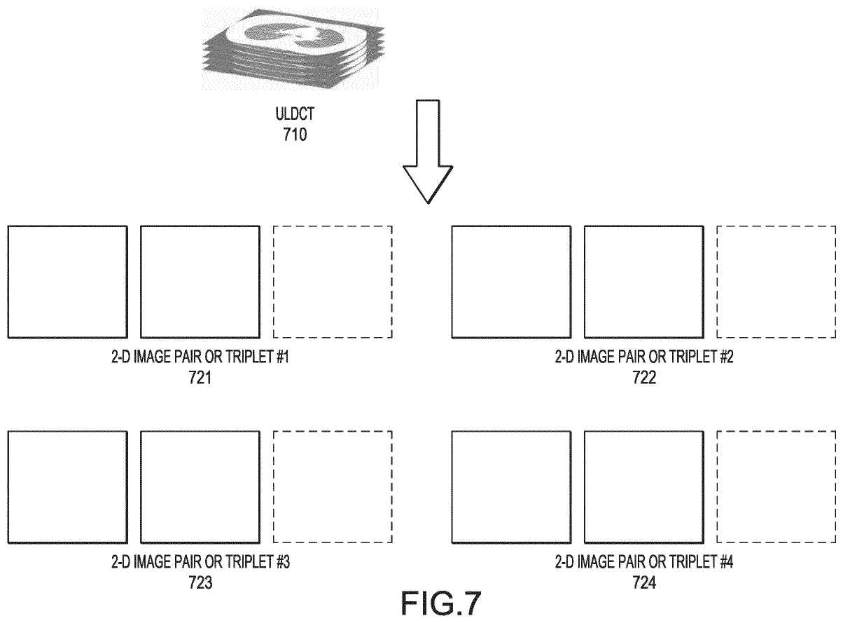

FIG. 7 illustrates a user interface for post-processing for radiological images, in accordance with a representative embodiment.

In FIG. 7, a ULDCT imaging scan 710 comprises a stack of radiological images. The two-dimensional image pair or triplet #1 721, the two-dimensional image pair or triplet #2 722, the two-dimensional image pair or triplet #3, and the two-dimensional image pair or triplet #4 are generated from cross-sectional data of the ULDCT imaging scan 710. The various two-dimensional image pairs and triplets in FIG. 7 are changeable and editable, so that a user may interactively obtain information sought by the user. For example, one or more images in the various two-dimensional image pairs and/or triplets may include an interactable region of interest indicator which indicates a region of interest. A region of interest may be a region in which artificial intelligence has detected a lesion, a broken bone, or another form of medical concern which is to be brought to the attention of a radiologist.

The system 100 from FIG. 1A may accept interactive instructions from the user such that further inspection of the region of interest may be designated by the user and accepted by the system 100. Further inspection of the region of interest results in (or may result in) generating a navigable subspace view through which the user may explore corresponding anatomical regions within the imaging scan in a magnified view compared to the generated plurality of two-dimensional images or subset thereof. In other words, selection of a region of interest indicator may result in a pop-up window or overlay or new view which shows the selected region of interest in a magnified view so that details in the selected region of interest may be more clearly seen. The region of interest indicator may be configured to enable one or more of acceptance, rejection or further inspection of the region of interest.

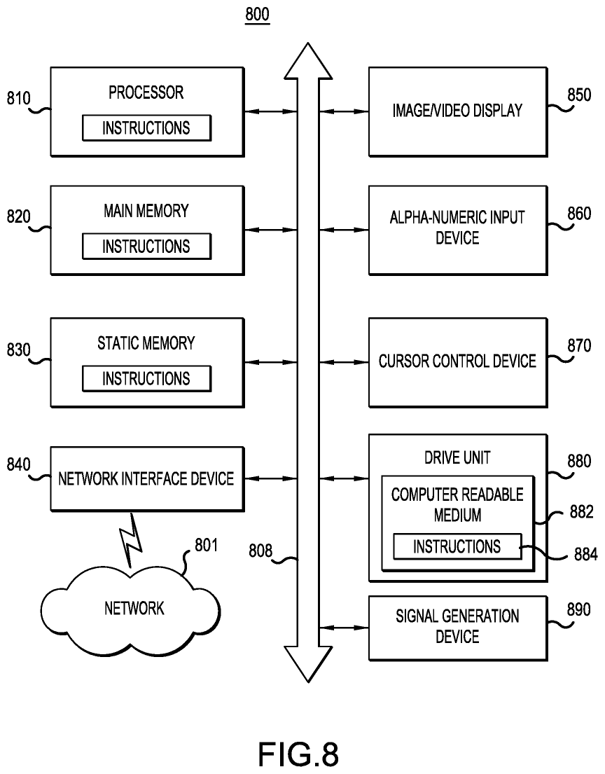

FIG. 8 illustrates a computer system, on which a method for post-processing for radiological images is implemented, in accordance with another representative embodiment.

Referring to FIG. 8, the computer system 800 includes a set of software instructions that may be executed to cause the computer system 800 to perform any of the methods or computer-based functions disclosed herein. The computer system 800 may operate as a standalone device or may be connected, for example, using a network 801, to other computer systems or peripheral devices. In embodiments, a computer system 800 performs logical processing based on digital signals received via an analog-to-digital converter.

In a networked deployment, the computer system 800 operates in the capacity of a server or as a client user computer in a server-client user network environment, or as a peer computer system in a peer-to-peer (or distributed) network environment. The computer system 800 may also be implemented as or incorporated into various devices, such as the computer 110, a workstation that includes the controller 150, a stationary computer, a mobile computer, a personal computer (PC), a laptop computer, a tablet computer, or any other machine capable of executing a set of software instructions (sequential or otherwise) that specify actions to be taken by that machine. The computer system 800 may be incorporated as or in a device that in turn is in an integrated system that includes additional devices. In an embodiment, the computer system 800 may be implemented using electronic devices that provide voice, video or data communication. Further, while the computer system 800 is illustrated in the singular, the term “system” shall also be taken to include any collection of systems or sub-systems that individually or jointly execute a set, or multiple sets, of software instructions to perform one or more computer functions.

As illustrated in FIG. 8, the computer system 800 includes a processor 810. The processor 810 may be considered a representative example of a processor of a controller and executes instructions to implement some or all aspects of methods and processes described herein. The processor 810 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 810 is an article of manufacture and/or a machine component. The processor 810 is configured to execute software instructions to perform functions as described in the various embodiments herein. The processor 810 may be a general-purpose processor or may be part of an application specific integrated circuit (ASIC). The processor 810 may also be a microprocessor, a microcomputer, a processor chip, a controller, a microcontroller, a digital signal processor (DSP), a state machine, or a programmable logic device. The processor 810 may also be a logical circuit, including a programmable gate array (PGA), such as a field programmable gate array (FPGA), or another type of circuit that includes discrete gate and/or transistor logic. The processor 810 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 800 further includes a main memory 820 and a static memory 830, where memories in the computer system 800 communicate with each other and the processor 810 via a bus 808. Either or both of the main memory 820 and the static memory 830 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 820 and the static memory 830 are articles of manufacture and/or machine components. The main memory 820 and the static memory 830 are computer-readable mediums from which data and executable software instructions may be read by a computer (e.g., the processor 810). Each of the main memory 820 and the static memory 830 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RANI memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 800 further includes a video display unit 850, such as a liquid crystal display (LCD), an organic light emitting diode (OLED), a flat panel display, a solid-state display, or a cathode ray tube (CRT), for example. Additionally, the computer system 800 includes an input device 860, such as a keyboard/virtual keyboard or touch-sensitive input screen or speech input with speech recognition, and a cursor control device 870, such as a mouse or touch-sensitive input screen or pad. The computer system 800 also optionally includes a disk drive unit 880, a signal generation device 890, such as a speaker or remote control, and/or a network interface device 840.

In an embodiment, as depicted in FIG. 8, the disk drive unit 880 includes a computer-readable medium 882 in which one or more sets of software instructions 884 (software) are embedded. The sets of software instructions 884 are read from the computer-readable medium 882 to be executed by the processor 810. Further, the software instructions 884, when executed by the processor 810, perform one or more steps of the methods and processes as described herein. In an embodiment, the software instructions 884 reside all or in part within the main memory 820, the static memory 830 and/or the processor 810 during execution by the computer system 800. Further, the computer-readable medium 882 may include software instructions 884 or receive and execute software instructions 884 responsive to a propagated signal, so that a device connected to a network 801 communicates voice, video or data over the network 801. The software instructions 884 may be transmitted or received over the network 801 via the network interface device 840.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, post-processing for radiological images enables a radiologist to quickly process results of an imaging scan comprising a stack of radiological images. The radiologist may refer to two-dimensional images generated from cross-sectional data of the imaging scan, and selectively control arranging, rearranging and editing of the generated two-dimensional images.

Although post-processing for radiological images has been described with reference to several exemplary embodiments, it is understood that the words that have been used are words of description and illustration, rather than words of limitation. Changes may be made within the purview of the appended claims, as presently stated and as amended, without departing from the scope and spirit of post-processing for radiological images in its aspects. Although post-processing for radiological images has been described with reference to particular means, materials and embodiments, post-processing for radiological images is not intended to be limited to the particulars disclosed; rather post-processing for radiological images extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A computer implemented method for reading an imaging scan, the method comprising:
accessing the imaging scan, the imaging scan comprising a stack of radiological images;
generating a plurality of two-dimensional images from cross-sectional data of the imaging scan, the plurality of two-dimensional images comprising projected information from the stack of radiological images, and the projected information comprising either a full imaged volume or an automatically-selected sub-volume and either a full range of image intensities or an automatically-selected sub-range of image intensities; and
displaying the plurality of two-dimensional images or a subset thereof in a user interface (UI) of an advanced interpretation environment, the user interface providing access to the stack of radiological images or additional information derived from the stack of radiological images, by enabling interaction with the generated plurality of two-dimensional images.

2. The computer implemented method of claim 1, further comprising:
accessing a profile data object corresponding to a patient of the imaging scan, the profile data object comprising patient data from one or more of a Radiology Information system (RIS), an HL7 broker, an Electronic Medical Record (EMR), a Picture Archive and Communication system (PACS), or meta information in the imaging scan; and
determining a submodule to process the cross-sectional data based on the accessed profile data object;
wherein generating the plurality of two-dimensional images further comprises processing, by the determined submodule, the cross-sectional data and the patient data, and the displayed plurality of two-dimensional images includes an interactable region of interest indicator which indicates a region of interest.

3. The computer implemented method of claim 2, wherein the region of interest indicator is configured to enable one or more of acceptance, rejection, or further inspection of the region of interest, wherein further inspection of the region of interest results in generating a navigable subspace view through which a user may explore corresponding anatomical regions within the imaging scan in a magnified view compared to the generated plurality of two-dimensional images or subset thereof.

4. The computer implemented method of claim 3, wherein the user interface further comprises a list of one or more measurements, one of the one or more measurements flagging an incidental finding and linking to an image location corresponding to the region of interest indicator.

5. The computer implemented method of claim 1, wherein the user interface further comprises a semi-transparent rendering of an object of interest overlaid on the generated plurality of two-dimensional images or subset thereof, the object of interest comprising one of lungs, a vasculature tree, or a respiratory tree.

6. The computer implemented method of claim 1, wherein the imaging scan is one of a computed tomography (CT) scan, a low dose CT (LDCT) an ultra-low dose CT (ULDCT) scan, a spectral or dual energy CT scan, a photon counting CT scan, a magnetic resonance (MR) scan, or a positron emission tomography (PET or PET-CT) scan.

7. The computer implemented method of claim 1, wherein generating the plurality of two-dimensional images further comprises executing one or more artificial intelligence processes on the stack of radiological images.

8. The computer implemented method of claim 7,
wherein the one or more artificial intelligence processes are configured to detect anatomical characteristics in the stack of radiological images, and
wherein the projected information from the stack of radiological images is derived based on an anatomical characteristic detected from the one or more artificial intelligence processes.

9. The computer implemented method of claim 1, further comprising:
selectively editing an object captured in the stack of radiological images,
wherein at least one of the plurality of two-dimensional images includes the selectively-edited object.

10. The computer implemented method of claim 1, wherein the plurality of two-dimensional images are editable to selectively display fewer than all types of anatomy captured in the stack of radiological images.

11. The computer implemented method of claim 1, further comprising:
automatically detecting an anatomical characteristic in the stack of radiological images, and
generating the plurality of two-dimensional images based on automatically detecting the detected anatomical characteristic in the stack of radiological images.

12. The computer implemented method of claim 11,
wherein the plurality of two-dimensional images are reconstruction images generated synthetically from the stack of radiological images and based on the detected anatomical characteristic.

13. A system for reading an imaging scan, comprising:
a memory that stores instructions; and
a processor that executes the instructions,
wherein, when executed by the processor, the instructions cause the system to:
access the imaging scan, the imaging scan comprising a stack of radiological images;
generate a plurality of two-dimensional images from cross-sectional data of the imaging scan, the plurality of two-dimensional images comprising projected information from the stack of radiological images, and the projected information comprising either a full imaged volume or an automatically-selected sub-volume and either a full range of image intensities or an automatically-selected sub-range of image intensities; and
display the generated plurality of two-dimensional images or a subset thereof in a user interface (UI) of an advanced interpretation environment, the user interface providing access to the stack of radiological images or additional information derived from the stack of radiological images, by enabling interaction with the generated plurality of two-dimensional images.

14. The system of claim 13, further comprising:
a display that provides the user interface for displaying the generated plurality of two-dimensional images or subset thereof.

15. A controller for reading an imaging scan, comprising:
a memory that stores instructions; and
a processor that executes the instructions,
wherein, when executed by the processor, the instructions cause a system which includes the controller to:
access the imaging scan, the imaging scan comprising a stack of radiological images;
generate a plurality of two-dimensional images from cross-sectional data of the imaging scan, the plurality of two-dimensional images comprising projected information from the stack of radiological images, and the projected information comprising either a full imaged volume or an automatically-selected sub-volume and either a full range of image intensities or an automatically-selected sub-range of image intensities; and
display on the display the generated plurality of two-dimensional images or a subset thereof in a user interface of an advanced interpretation environment, the user interface providing access to the stack of radiological images or additional information derived from the stack of radiological images, by enabling interaction with the generated plurality of two-dimensional images.
